(19) United States
(12) Patent Application Publication
(10) Pub. No.: US 2018/0117271 A1
(43) Pub. Date: May 3, 2018

(54) SYSTEM FOR PNEUMATIC TESTING OF GAS FLOW MODULE AND METHOD OF OPERATION THEREOF

(71) Applicant: KONINKLIJKE PHILIPS N.V., EINDHOVEN (NL)

(72) Inventors: ERIC PAUL WIGFORSS, WALLINGFORD, CT (US); PAUL BRUCE GUNNESON, CHESHIRE, CT (US)

(21) Appl. No.: 15/553,187

(22) PCT Filed: Feb. 25, 2016
(86) PCT No.: PCT/EP2016/053936

§ 371 Date: Aug. 24, 2017

Related U.S. Application Data
(60) Provisional application No. 62/120,601, filed on Feb. 25, 2015.

(51) Int. Cl. A61M 16/00; G01M 3/26; A61M 16/20

(52) U.S. Cl. CPC A61M 16/024; G01M 3/26; A61M 16/202; A61M 2016/0027; A61M 2209/02; A61M 2205/15; A61M 2205/6018; A61M 2205/70; A61M 2016/0033

(57) ABSTRACT

A gas flow monitoring system (100, 200A, 200B, 400, 600, 700) for monitoring a patient gas flow and having pressure sensors (114), a pneumatic system (110), valves (120), and a pump (116), the system including a controller (112, 710) configured to: determine whether a test mode is selected; configure the pump and the valves to pressurize at least a portion of the pneumatic system when in a test mode; obtain sensor information indicating pressure within at least a portion of the pneumatic system, when in the test mode; and determine whether a leak test fails based upon at least the sensor information.

The present system relates to a system for monitoring the performance of a gas flow module which determines flow characteristics of a gas flow for example to a system for performing automated pneumatic testing of a gas flow module such as a respiration gas flow module, and a method of operation thereof.

Typically, ventilation systems provide a ventilation gas mixture to mechanically ventilate a patient coupled thereto. Characteristics of this gas mixture such as volume, flow, and pressure can be determined using a flow sensor. Conventional flow sensors incorporate a pneumatic system that can leak and, as such, require manual testing using a suitable tester such as a manually operated syringe on a periodic basis to detect leaks. Unfortunately, manual testing of pneumatic systems is difficult and time consuming to perform, especially when performed in the field. For example, conventional pneumatic system testing methods which use the manually-operated syringe require a user to manually manipulate the syringe to pressurize a pneumatic system and thereafter manually check for leaks. Unfortunately, this test can only detect a single failure mode and results that can often vary. Accordingly, embodiments of the present system may overcome these and other disadvantages in the prior art systems.

The system(s), device(s), method(s), arrangements(s), user interface(s), computer program(s), processes, etc. (hereinafter each of which will be referred to as system, unless the context indicates otherwise), described herein address problems in prior art systems.

In accordance with embodiments of the present system, there is disclosed a gas flow monitoring system for monitoring a gas flow such as a ventilation gas flow. The gas flow monitoring system may include at least one port, pressure sensors, a pneumatic system, valves, and a pump, such as a suitable automatic system for creating pneumatic flow, vacuum and/or pressure. The system may include a controller configured to: control the pump and the valves to pressurize at least a portion of the pneumatic system when in a test mode; obtain sensor information indicating pressure within at least a portion of the pneumatic system; and determine whether a leak test fails based upon at least the sensor information. In accordance with embodiments, the controller may be configured to render results of the determination indicating whether the leak test has failed. The at least one port may include a gas flow portion, for example positioned proximal to the patient though in accordance with embodiments of the present system, may be positioned otherwise. Further, the gas flow monitoring system may determine whether a test mode is selected, and may enter the test mode when it is determined that the test mode is selected.

In accordance with embodiments, the controller may be configured to determine whether an accessory is coupled to the at least one port. The controller may be configured to determine a type of accessory when it is determined that an accessory is coupled to the at least one port. The accessory may include identification information which identifies a type of the accessory. The controller may be configured to read the identification information from the accessory portion. The controller may be configured to identify a type of accessory based upon the identification information. The controller may be configured to determine that the test mode is selected when the type of accessory is determined to be a test type. The controller may be configured to determine whether a breath analysis mode is selected based on the identified type of accessory. The controller may further be configured to control the pump and the valves to obtain a sample gas flow from a patient interface when it is determined that a breath analysis mode is selected.

In accordance with embodiments of the present system, the controller may be configured to render results of the determination of whether the leak test fails. When the breath analysis mode is selected, the controller may be configured to determine whether a recent test mode has previously failed. The controller may be configured to terminate the breath analysis mode when it is determined that the recent test mode has previously failed.

In accordance with embodiments, there is disclosed a method for monitoring a gas flow such as a ventilation gas flow in a system having pressure sensors, a pneumatic system, valves, and a pump. The method may include acts of: configuring the pump and the valves to pressurize at least a portion of the pneumatic system when in a test mode; obtaining sensor information indicating pressure within at least a portion of the pneumatic system, when in the test mode; and determining whether a leak test fails based upon at least the sensor information. The method may include acts of determining whether an accessory is coupled to the at least one port; and determining a type of accessory when it is determined that an accessory is coupled to the at least one port. The method may include an act of determining that the test mode is selected when the type of accessory is determined to be a test type.

In accordance with embodiments, there is disclosed a computer readable non-transitory medium having computer readable program code for operating on a computer for performing a method of monitoring a gas flow in a system having at least one port, pressure sensors, a pneumatic system, valves, and a pump, the method comprising acts configuring the pump and the valves to pressurize at least a portion of the pneumatic system when in a test mode; obtaining sensor information indicating pressure within at least a portion of the pneumatic system, when in the test mode; and determining whether a leak test fails based upon at least the sensor information. The medium may include computer readable program code for determining whether an accessory is coupled to the at least one port; and determining a type of accessory when it is determined that an accessory is coupled to the at least one port. The medium may include computer readable program code for determining that the test mode is selected when the type of accessory is determined to be a test type.

The present invention is explained in further detail in the following exemplary embodiments and with reference to the figures, where identical or similar elements are partly indicated by the same or similar reference numerals, and the features of various exemplary embodiments being combinable. In the drawings:

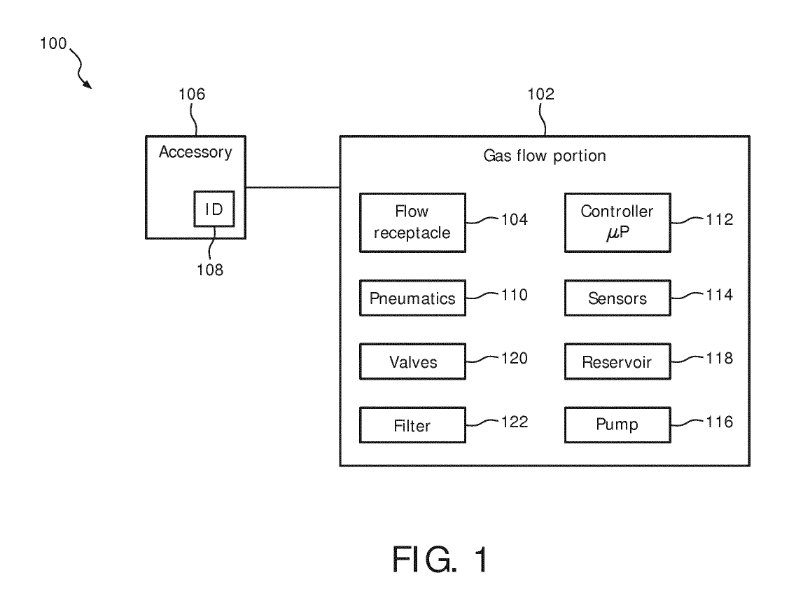

FIG. 1 shows a portion of a block diagram of a sensor system operating in accordance with embodiments of the present system;

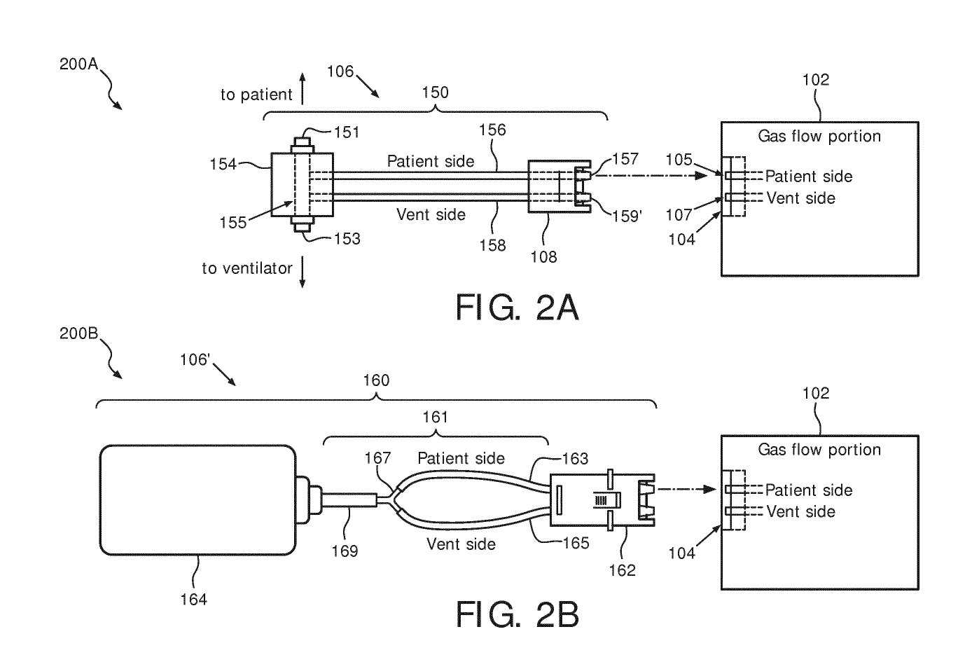

FIG. 2A shows a portion of an exploded block diagram of a proximal flow portion coupled to an accessory including a patient-type interface operating in accordance with embodiments of the present system;

FIG. 2B shows a portion of an exploded block diagram of a proximal flow portion coupled to an test-type portion including a test interface operating in accordance with embodiments of the present system;

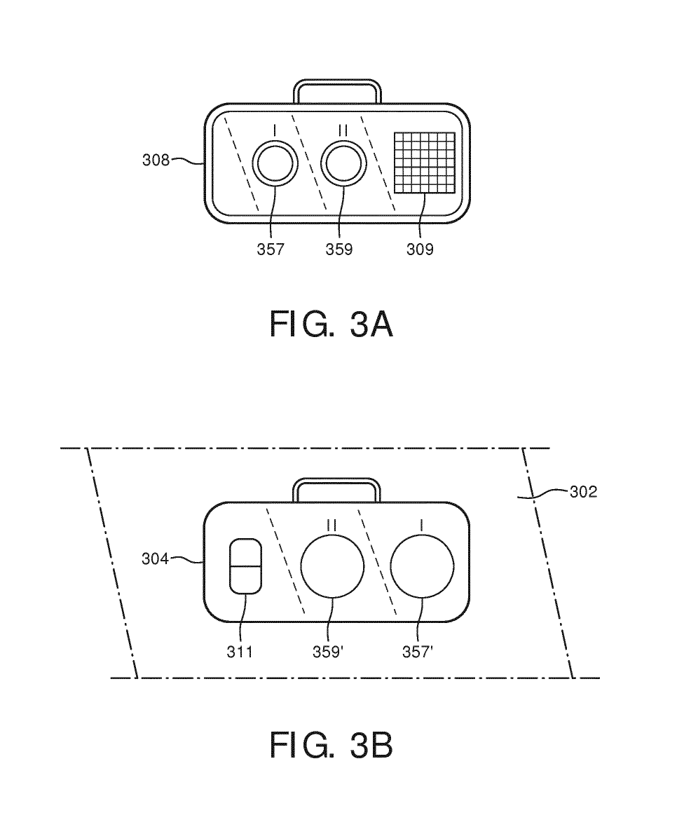

FIG. 3A shows a portion of front side view of a proximal flow receptacle in accordance with embodiments of the present system;

FIG. 3B shows a portion of a front side view of a flow receptacle of a proximal flow portion in accordance with embodiments of the present system;

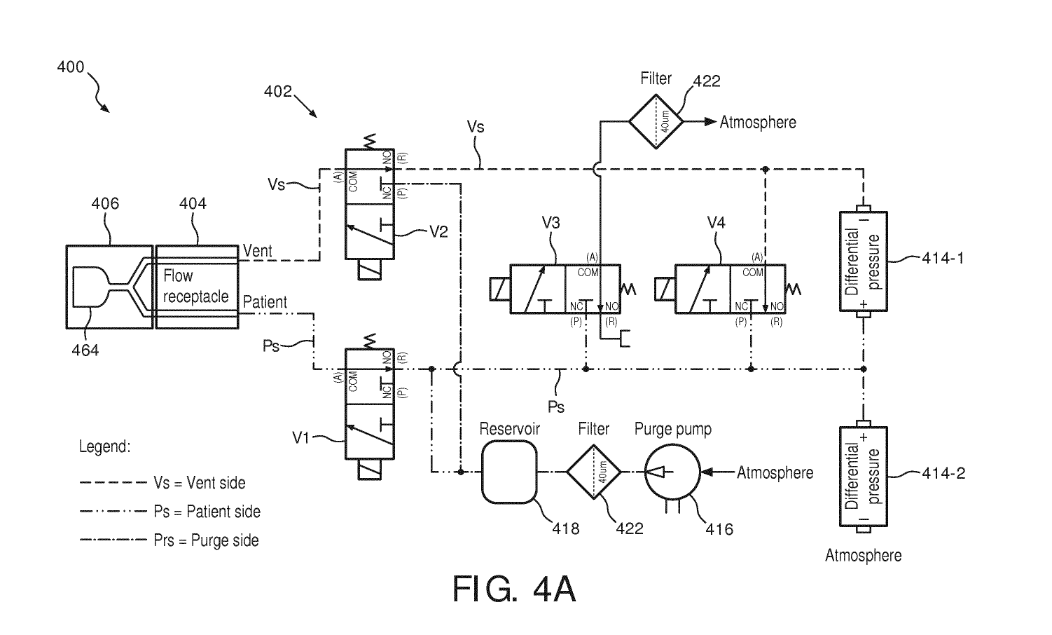

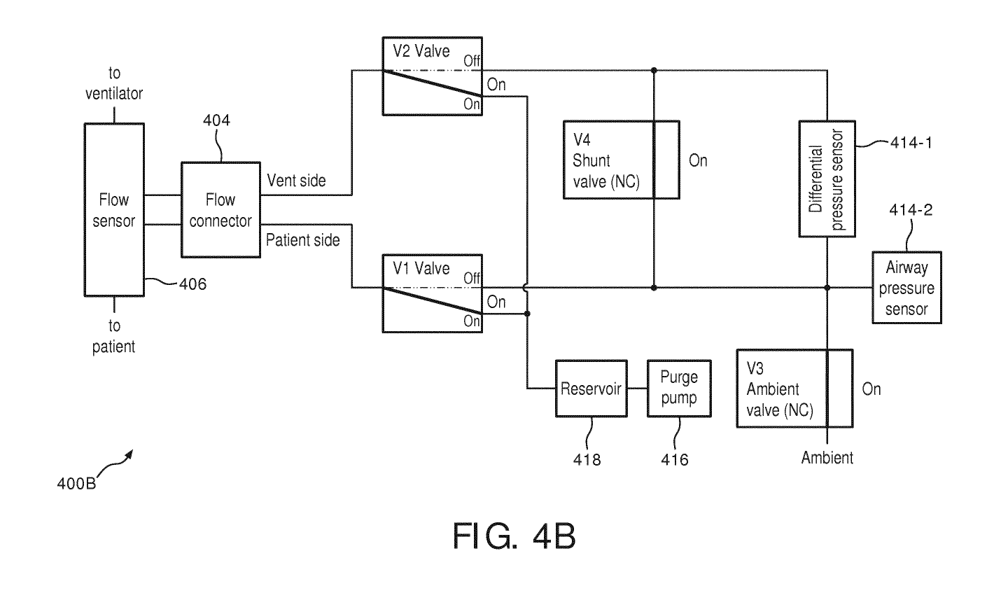

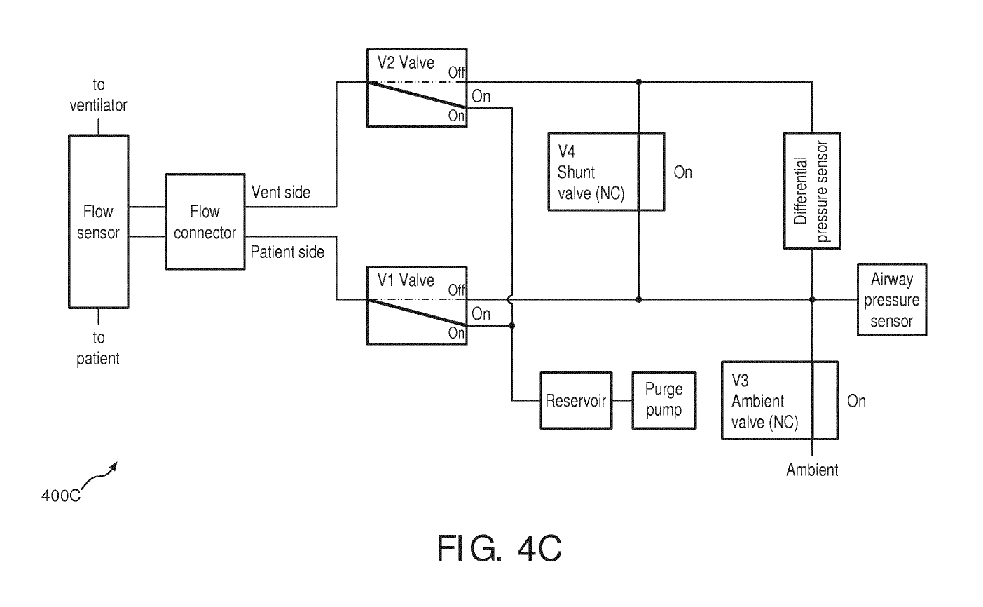

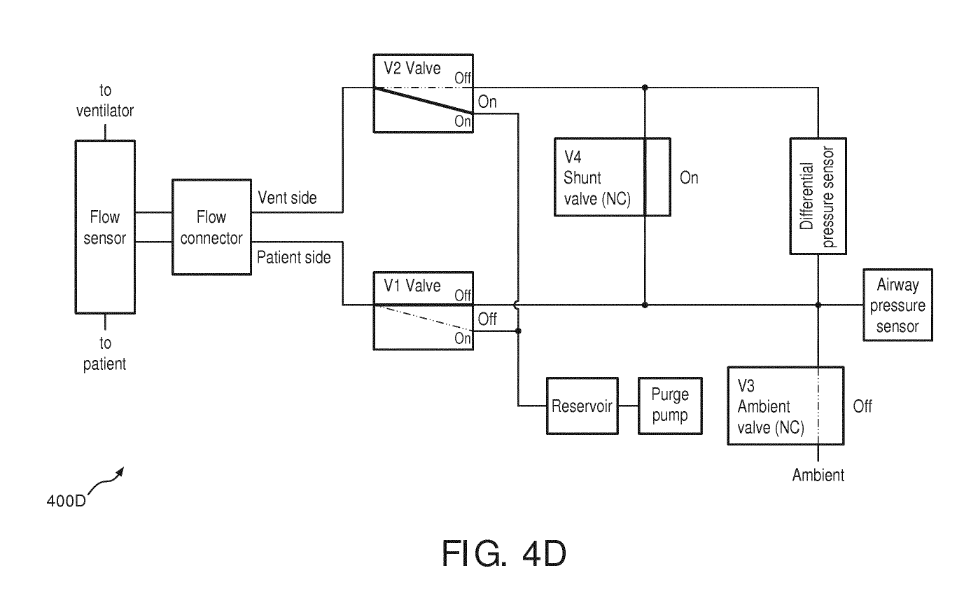

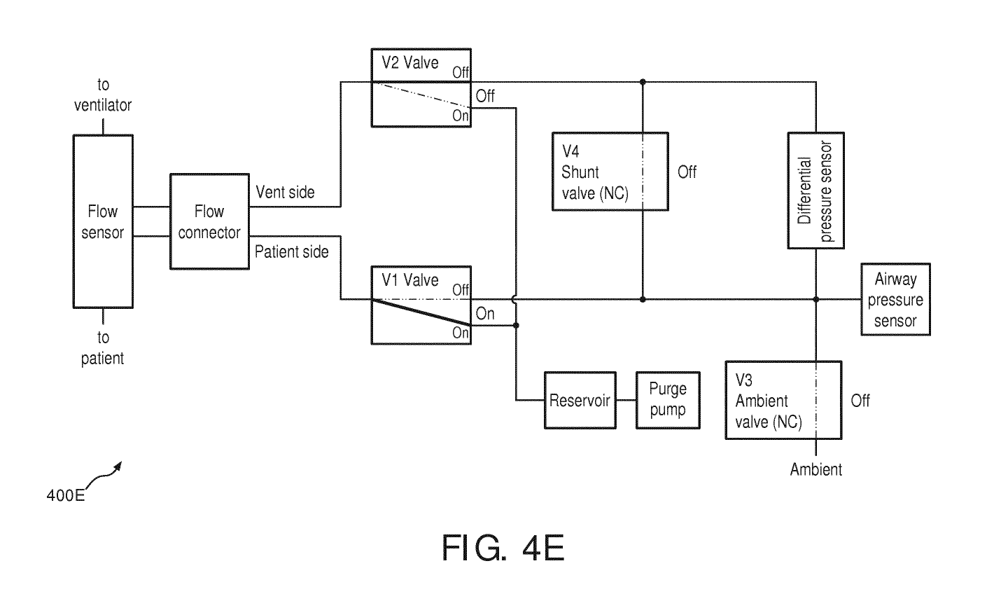

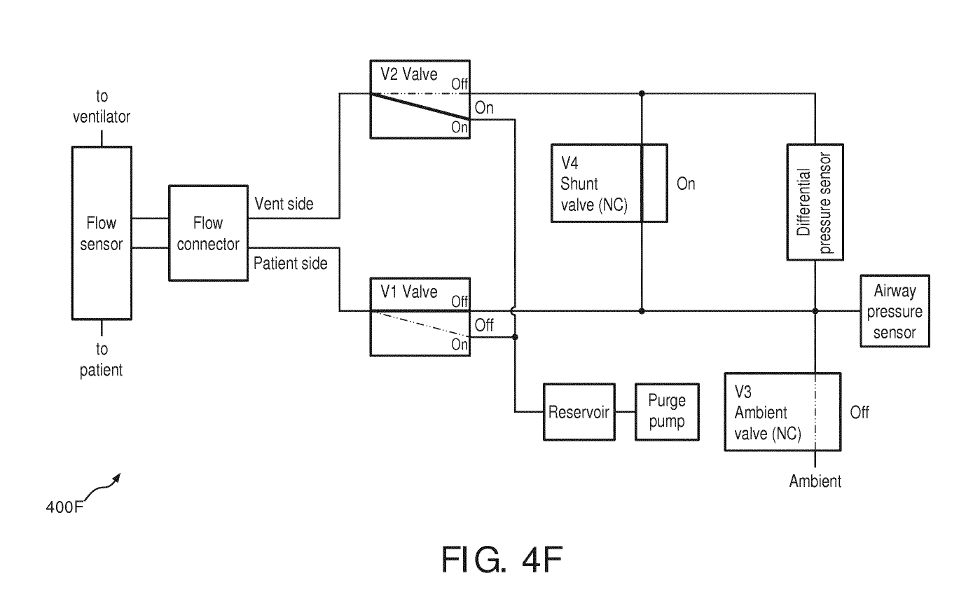

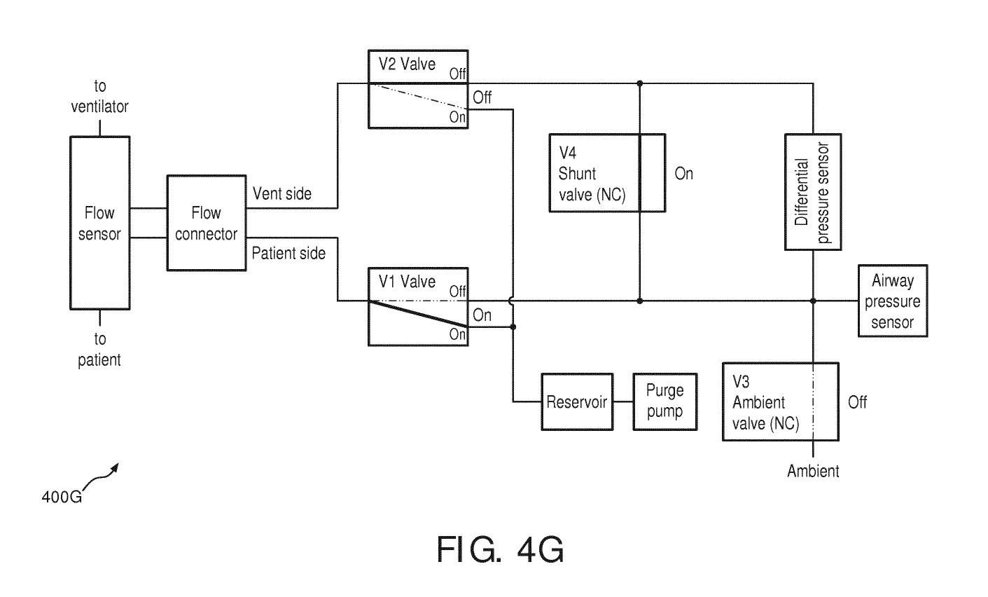

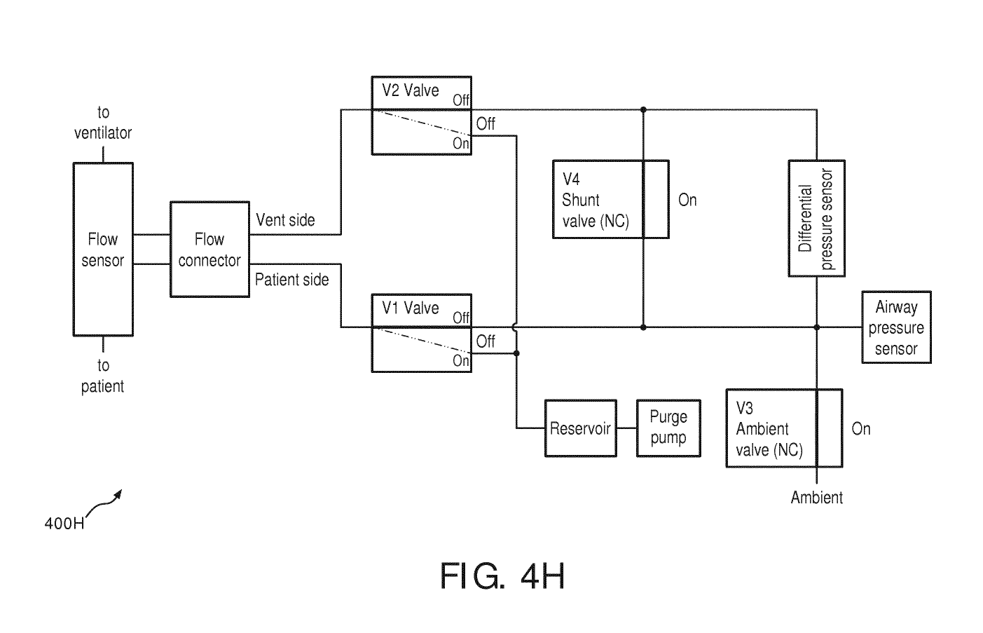

FIG. 4A shows a portion of a block diagram of a system operating in accordance with embodiments of the present system;

FIG. 4B shows a portion of a block diagram of a system that is similar to the system of FIG. 4A operating in accordance with embodiments of the present system;

FIG. 4C shows a portion of a block diagram of a system that is similar to the system of FIG. 4A operating in accordance with embodiments of the present system;

FIG. 4D shows a portion of a block diagram of a system that is similar to the system of FIG. 4A operating in accordance with embodiments of the present system;

FIG. 4E shows a portion of a block diagram of a system that is similar to the system of FIG. 4A operating in accordance with embodiments of the present system;

FIG. 4F shows a portion of a block diagram of a system that is similar to the system of FIG. 4A operating in accordance with embodiments of the present system;

FIG. 4G shows a portion of a block diagram of a system that is similar to the system of FIG. 4A operating in accordance with embodiments of the present system;

FIG. 4H shows a portion of a block diagram of a system that is similar to the system of FIG. 4A operating in accordance with embodiments of the present system;

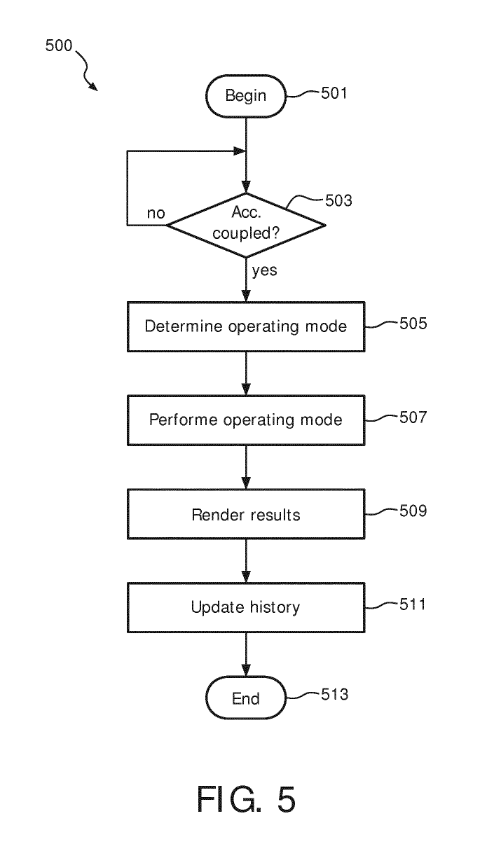

FIG. 5 shows a functional flow diagram performed by a process in accordance with embodiments of the present system;

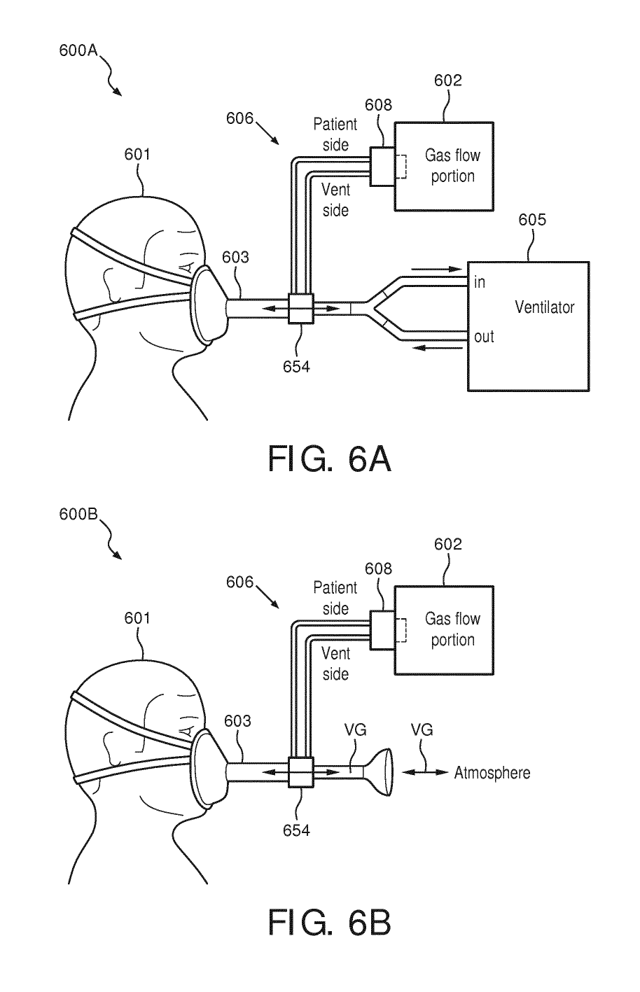

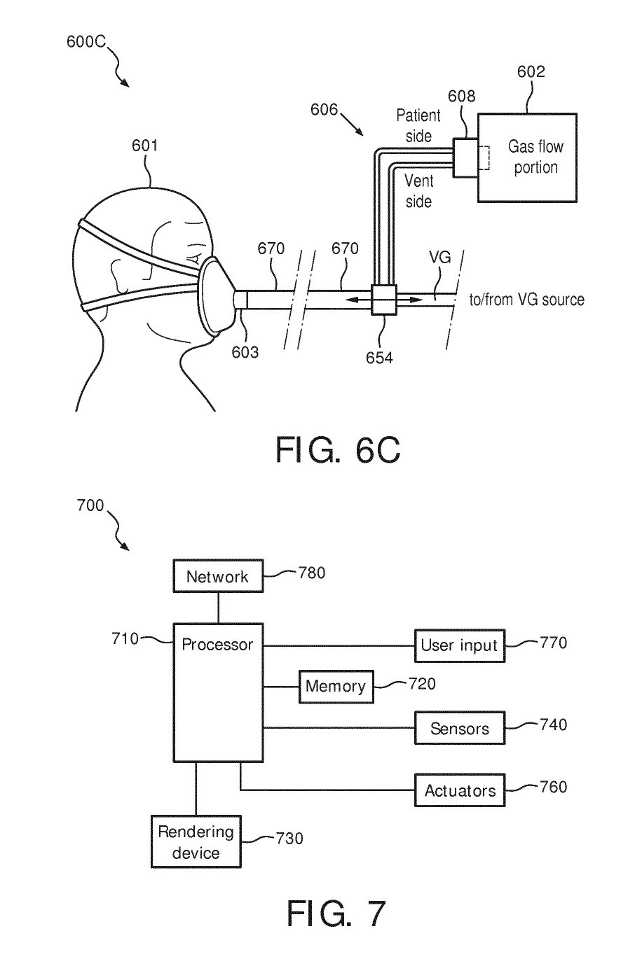

FIG. 6A shows a block diagram of a portion of the present system operating in a ventilation detection mode in accordance with embodiments of the present system;

FIG. 6B shows a block diagram of a portion of a patient gas flow system operating in accordance with embodiments of the present system;

FIG. 6C shows a block diagram of a portion of a patient gas flow system 600C operating in accordance with embodiments of the present system; and

FIG. 7 shows a portion of a system in accordance with embodiments of the present system.

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well known devices, circuits, tools, techniques, and methods are omitted so as not to obscure the description of the present system. It should be expressly understood that the drawings are included for illustrative purposes and do not represent the entire scope of the present system. In the accompanying drawings, like reference numbers in different drawings may designate similar elements.

FIG. 1 shows a portion of a block diagram of a sensor system 100 (hereinafter system 100 for the sake of clarity) operating in accordance with embodiments of the present system. The system 100 may include any suitable gas-flow sensor such as a gas flow sensor which may monitor pressure, flow, and/or volume of a gas (such as air, a ventilation gas, etc.) provided to a subject (e.g., an animal, a human, etc.) hereinafter a patient for the sake of simplicity. For example, the sensor system 100 may include a FloTrak Elite™ respiratory mechanics module by Philips Respironics which may be coupled to a physical interface. In accordance with embodiments of the present system, the physical interface may be coupled to system to supply a gas mixture to a patient and/or the physical interface may be coupled to atmosphere (e.g., an atmospheric source) so as to receive an ambient gas such as air as a gas mixture.

The sensor 100 may include a gas flow portion (PFP) 102 having a flow receptacle portion (FRP) 104 that may be shaped and/or sized or otherwise configured to be coupled to an accessory (AP) 106 as described herein. In accordance with embodiments of the present system, the gas flow sensor may be coupled or otherwise positioned proximate to a patient for supplying a gas flow to the patient. In accordance with further embodiments the gas flow sensor may be otherwise positioned (e.g., not be proximal to the patient).

The accessory 106 may include a gas flow receptacle and an identification (ID) portion 108. The ID portion may include ID information related to characteristics of the accessory 106 such as, for example, one or more of serial number, type, date of manufacture, operating parameters, etc. The serial number may identify a serial number of the accessory 106 or portions thereof. The type ID may identify the accessory's type (e.g., type 1=test portion, type 2=patient interface, etc.). The ID information may be stored in any suitable format such as in a memory of the system such as a memory of the accessory 106 so that the ID information may be read by the gas flow portion 102 for example when coupled together and/or otherwise when desired, programmed, etc. With regard to the type ID information, this information may identify a type of the interface portion such as a patient-type interface (PTI) or a test-type interface (TTI) as illustratively described herein. Although two different types of interface portions are described herein, it should be understood that other types of interfaces such as may be defined by the system, process and/or user are also contemplated in accordance with embodiments of the present system.

For example, FIG. 2A shows a portion of an exploded block diagram of a system 200A including a gas flow portion 102 coupled to an accessory 106 including a patient-type interface 150 operating in accordance with embodiments of the present system. FIG. 2B shows a portion of a exploded block diagram of a system 200B including a gas flow portion 102 coupled to an accessory 106 including a test-type interface 160 operating in accordance with embodiments of the present system.

With reference to FIG. 2A, the patient-type interface 150 may include proximal and distal flow receptacles 108 and 154, respectively, which may have two or three pneumatic ports each and which may be coupled to each other by one or more pneumatic hoses 156, 158 and may be separate from or formed integrally with each other. The gas flow receptacle 108 may be shaped and/or sized to so that it may be coupled to a receptacle 104 of the gas flow portion 102. The receptacle 104 of the gas flow portion 102 may include patient and vent side pneumatic openings 105 and 107, respectively. The distal flow receptacle 154 may include a tube 155 configured to flow couple a ventilator to a patient interface. Accordingly, the tube 155 may include first and second openings 151 and 153, respectively. The first opening 151 may be coupled to a physical interface such as an intrusive-type physical interface (e.g., intubation tube, etc.) or a non-intrusive-type physical interface (e.g., a mask, nasal cannula, etc.) that may be coupled to the patient. The second pneumatic port 153 may be coupled to a ventilator which may provide ventilation gas to the patient. In accordance with embodiments of the present system, the patient interface 150 may have an ID that for example may identify the patient interface 150 as a patient interface (e.g., a patient-type interface).

With reference to FIG. 2B, the test-type interface 160 may include proximal and distal flow receptacles 162 and 164, respectively, which may be coupled to each other by a coupler 161. The gas flow receptacle 164 may be similar to the gas flow receptacle 108. However, in accordance with embodiments of the present system an ID may identify the test-type interface 160 as a test-type interface (e.g., non-patient-type interface). With regard to the distal flow receptacle 164, this receptacle may include a reservoir having a desired volume which may be selected by the system and/or a user. For example, it has been observed during testing that a volume of 250 ml may provide satisfactory results. However, in accordance with embodiments of the present system other volumes are also envisioned. With regard to the coupler 161, this coupler may include first through third hoses 163, 165, and 169 which may be coupled to each other for example using a wye-type connector 167 situated between the reservoir and the gas flow receptacle 162 (e.g., along a flow path) so that the first through third hoses 163, 165, and 169 may be flow coupled to each other. The first hose 163 may be a patient side hose and the second hose 165 may be a vent side hose. Accordingly, a common-mode tie may be used to couple the distal flow receptacle to the first and second pneumatic portions (which may be patient and vent side portions, respectively).

In accordance with embodiments of the present system the gas flow receptacles (e.g., 108 and 162) may be shaped and/or sized so as to form a mechanical key which may identify a type of the corresponding interface (e.g., test-type interface or patient-type interface). This mechanical key may then trigger a sensor (e.g., a micro switch) to indicate its presence and/or identify the interface type (e.g., patient-type, test-type). For example, the gas flow receptacle 162 of the test-type interface 160 may be shaped and/or sized to trigger a first micro-switch and the gas flow receptacle 108 of the patient-type interface 150 may be shaped and/or sized to trigger a second micro-switch when coupled to the receptacle portion 104 of the gas flow portion 102. In accordance with embodiments of the present system, a controller of the system may utilize the mechanical key, such as through operation of the micro-switches, to determine whether a gas flow receptacle is coupled to the receptacle 104 of the gas flow portion 102 and/or may determine the type of the corresponding interface (e.g., patient-type or test-type interface) such as based upon which micro-switch is triggered and/or not triggered. Accordingly, these sensors (e.g., micro switches) may provide sensor information to the controller for operation in accordance with embodiments of the present system.

Referring back to FIG. 1, the gas flow portion 102 may include one or more of the flow receptacle portion (FRP) 104, a pneumatics portion 110, a controller 112, sensors 114, a pump 116, a reservoir 118, valves 120, and a filter 122 one or more of which may be fluidically coupled to each other and/or may otherwise communicate with each other.

The flow receptacle portion 104 may include flow ports 108-1-108-N (generally 108-x) (where N is an integer greater than 1 such that there may be two or more flow ports which may be coupled to a pneumatics portion 110 of the gas flow portion 102.

In accordance with embodiments of the present system, the controller 112 may control the overall operation of the gas flow portion 102. For example, the sensors 114 may be operative under the control of the controller 112 to detect conditions such as one or more of temperature, pressure (e.g., differential pressure), and flow external to and/or within the pneumatic portion 110 and form corresponding sensor information which may be provided to the controller 112 for further processing.

In accordance with embodiments, the sensors 114 may further include identification (ID) sensors such as mechanical, electrical, magnetic and/or optical sensors which may detect an identification (ID) of the accessory 106. The ID of the accessory 106 may include desired information such as a serial number, a type (e.g., test accessory, gas sampling accessory, etc.), etc. Accordingly, the sensors 114 may determine an ID of the accessory 106, form corresponding ID information, and provide this ID information to the controller 112 for further processing. In this way, the controller 112 utilizing the ID information may then determine a type of the accessory (106, 106′).

The pump 116 may include one or more pumps operative under the control of the controller 112 to pump gasses to and/or from the pneumatic portion 110 coupled thereto. Accordingly, the pump 116 may pump gasses to and/or from the pneumatics portion 110 to, for example, pressurize and/or evacuate the pneumatics portion 110. For example, in accordance with embodiments, the pump 116 may pump atmospheric air and/or supplemented gases (e.g., one or more of supplemental, oxygen, nitrogen, water vapor, etc.) into the pneumatics portion 110 under the control of the controller 112. In yet other embodiments, the pump 116 may evacuate gasses within the pneumatics portion 110 and discharge the evacuated gases to a desired location and/or into the atmosphere. The pump 116 may include any suitable pump which may generate a pneumatic flow, pressure, and/or vacuum under the control of the controller 112.

The reservoir 118 may be shaped and/or sized so as to have a desired interior volume which may form one or more reservoirs for gasses within the pneumatics portion 110 coupled thereto. In accordance with embodiments, the reservoir may be integrally located within a conduit of the pneumatics portion 110 and/or may be distributed, if desired.

The valves 120 may be coupled to the pneumatics portion 110 and may be operative under the control of the controller 112 to control the flow of gasses within the pneumatics portion 110. The valves 120 may include any suitable valves such as solenoid operated pneumatic valves or the like. The filter 122 may include one or more filters coupled to the pneumatics portion 110 and which may be operative to condition gasses provided to the filter 122 and output conditioned gas. The conditioning may include, for example, filtering, drying, etc., as may be desired.

FIG. 3A shows a portion of front side view of a gas flow receptacle 308 in accordance with embodiments of the present system. The gas flow receptacle 308 may be similar to the gas flow receptacles 108 or 162 however, an identification portion may differ. For example, an ID portion 309 may include an optical identifier such as a QR code or any other suitable identifier. First and second pneumatic ports 357 and 359 may correspond with patient and vent side ports, respectively.

FIG. 3B shows a portion of a front side view of a flow receptacle portion 304 of a gas flow portion 302 in accordance with embodiments of the present system. The gas flow portion 302 may be similar to the gas flow portion 102. Accordingly, the flow receptacle portion 304 may be similar to the flow receptacle portion 104. However, the flow receptacle portion 304 may include an optical sensor which may read the QR code of the ID portion 309 of the gas flow receptacle 308 and provide corresponding sensor information including ID information to a controller of the system. First and second pneumatic ports 357′ and 359′, respectively, may correspond with the patient and vent side ports, respectively, and may be configured to be coupled to the respective ports of the gas flow receptacle 308 such as the first and second pneumatic ports 357 and 359, respectively.

FIG. 4A shows a portion of a block diagram of a system 400 operating in accordance with embodiments of the present system. The system 400 may include a gas flow portion that may be similar to the system 100. Accordingly, the system 400 may include a gas flow portion 402 and an accessory 406. The gas flow portion 402 may include one or more of valves v1 through v4, sensors such as differential pressure and airway pressure sensors 414-1 and 414-2, respectively, (generally pressure sensors 414-x), a reservoir 418, filters such as filters 422, a pump such as a purge pump 416, and a flow receptacle portion 404, which may be similar to one or more of the valves 120, the sensors 114, the reservoir 118, the filter 122, the pump 116, and the flow receptacle portion 104, respectively, of the system 100. The system 400 may include two or more pneumatic circuits such as a vent side circuit (VS) and a patient side circuit (PS). The vent side circuit (VS) may be coupled to a vent port (vent) of the flow receptacle portion 404 and the PS side may be coupled to a patient port (patient) of the flow receptacle portion 404. The pressure sensors 414-x may include transducer-type pressure sensors.

The accessory 406 may include a test-type accessory such as may be coupled to the flow receptacle portion 404. For example, assuming the accessory is a test-type accessory it may include a reservoir 464 which is similar to the reservoir 164 as shown in FIG. 2B may be coupled to the vent side circuit (VS) and the patient side circuit (PS).

FIGS. 4B through 4H shown below each illustrate a circuit configuration that is similar to (e.g., equivalent to) the circuit of FIG. 4A and may be configured in accordance with circuit operations which may be described for example with respect to the description of FIG. 5 below. Accordingly, similar reference numerals may be provided to illustrate similar portions.

Generally, FIGS. 4B-4E illustrate embodiments and/or modes wherein a flow sensor and/or patient interface may be connected to the flow connector. For example, FIG. 4B shows a portion of a block diagram of a system 400B that is equivalent to the system 400A of FIG. 4A operating in accordance with embodiments of the present system. FIG. 4B illustrates an embodiment of a normal operating configuration used in normal operating conditions with valves V1, V2 “OFF” (e.g., turned off) to measure patient airway flow and patient airway pressure. The differential pressure measured by the differential pressure sensor 414-1 may be utilized to determine patient airway flow calculations. The airway pressure sensor 414-2 may be utilized in this configuration to measure the patient airway pressure.

FIG. 4C shows a portion of a block diagram of a system 400C that is similar to the system 400A of FIG. 4A operating in accordance with embodiments of the present system though the similar individual elements are not further labeled so as not to obscure the description of the present system. This figure illustrates a configuration that for example may be utilized to zero the differential pressure and airway pressure sensors. As shown, valves V1 and V2 are turned on to isolate the differential pressure and airway pressure sensor from the patient side. Further valve V4 is turned on to equalize the differential pressure measured across the differential pressure sensor (e.g., simulating a no flow condition). Valve V3 is turned on so the airway pressure measured by the airway pressure sensor is zero (e.g., ambient). When the valves are configured as shown, the differential pressure and airway pressure measurements measured by the corresponding sensors are set to zero baseline measurements. During Normal Operation, the differential pressure and airway pressure sensor measurements are the delta from the zero measurements (e.g., difference between a given sensor measurement and the zero baseline measurement is provided as the sensor measurement).

FIG. 4D shows a portion of a block diagram of a system 400D that is similar to the system 400A shown in FIG. 4A operating in accordance with embodiments of the present system that may be utilized to purge the ventilator side such as tubing and/or valves. This figure illustrates a configuration that may be utilized to purge the flow sensor tubing ventilator side with the valve V2 turned on fluidically connecting the purge pump to the ventilator side. In this configuration the purge pump is turned on and gas flows down the flow sensor tubing on the ventilator side of the flow sensor. Purging as illustrated may be utilized to reduce or eliminate water that has collected in the flow sensor tubing during monitoring on the ventilator side.

FIG. 4E shows a portion of a block diagram of a system 400E that is similar to the system 400A of FIG. 4A operating in accordance with embodiments of the present system that may be utilized to purge the patient side such as tubing and/or valves. This figure illustrates a configuration that may be utilized to purge the patient side including the flow sensor tubing, etc. In this configuration, the purge pump is turned on and gas flows down the flow sensor tubing on the patient side of the flow sensor. Purging as illustrated may be utilized to reduce or eliminate water that has collected in the flow sensor tubing during monitoring on the patient side.

FIGS. 4F-4H show embodiments and/or modes wherein the flow sensor such as previously shown may be replaced with a leak test sensor hookup. In accordance with embodiments, the leak test sensor may be a volume, such as a 125 ml volume/reservoir where both sides of the flow sensor connect to the reservoir. In accordance with embodiments, the purge pump which is used in normal operation to periodically purge the flow sensor tubing for example may be utilized to charge the reservoir to a set pressure. Once the reservoir is charged to a pressure, the pressure sensor in the flow system (e.g., the airway pressure sensor) may be utilized to perform measurement(s) to determine whether or not portions of the system are failing, such as to determine whether there is leakage in the system. In accordance with embodiments, handy leak tests are provided that can test for system leaks in the field without a need to introduce addition sensor elements, though as may be appreciated, additional sensors may be provided. In accordance with embodiment, a leak test may also test that the purge pump is working properly.

FIG. 4F shows a portion of a block diagram of a system 400F that is similar to the system 400A of FIG. 4A operating in accordance with embodiments of the present system that may be utilized to perform a leak test of valve V2 and thereby, determine whether valve V2 is functioning properly. In this configuration, the purge pump may be turned on to charge the leak test sensor reservoir via valve V2 to a specified (e.g., default and/or user settable) pressure. In the accordance with embodiments, the pump may be left on until the specified pressure is measured by the airway pressure sensor. In accordance with embodiments, this test may indicate a failure in a case wherein a maximum charge time (e.g., default and/or user settable) is exceeded. In accordance with embodiments, valve V4 is turned on to equilibrate the pressure across the differential pressure sensor.

As stated, in a case wherein the airway pressure sensor does not charge to the specified airway pressure (e.g., the airway sensor does not reach and/or maintain a predetermined pressure indication), then the leak test may be determined to have failed and an indication to that effect may be rendered as described herein. Further, once the system is charged to the specified pressure, in accordance with embodiments, to allow the system pressure to settle the system may wait a period of time, such as 10 seconds. After the period, the pressure may be monitored for a further period of time to determine whether the pressure drops in the further period (e.g., 10 seconds). For example, in a case wherein the pressure drops, for example by more than 15 cmH2O in 10 seconds, then the leak test may be determined to have failed and an indication to that effect may be rendered such as “LEAK TEST OF VALVE V2 HAS FAILED”.

FIG. 4G shows a portion of a block diagram of a system 400G that is similar to the system 400A of FIG. 4A operating in accordance with embodiments of the present system that may be utilized to perform a leak test of valve V1 and for example, thereby determine whether valve V1 is functioning properly. In this configuration, the purge pump may be turned on to charge the leak test sensor reservoir via valve V1 to a specified (e.g., default and/or user settable) pressure. In the accordance with embodiments, the pump may be left on until the specified pressure is measured by the airway pressure sensor. In accordance with embodiments, this test may indicate a failure in a case wherein a maximum charge time (e.g., default and/or user settable) is exceeded. In accordance with embodiments, valve V4 is turned on to equilibrate the pressure across the differential pressure sensor.

As stated, in a case wherein the airway pressure sensor does not charge to the specified airway pressure (e.g., the airway sensor does not reach and/or maintain a predetermined pressure indication), then the leak test may be determined to have failed and an indication to that effect may be rendered as described herein. Further, once the system is charged to the specified pressure, in accordance with embodiments, to allow the system pressure to settle the system may wait a period of time, such as 10 seconds. After the period, the pressure may be monitored for a further period of time to determine whether the pressure drops in the further period (e.g., 10 seconds). For example, in a case wherein the pressure drops, for example by more than 15 cmH2O in the 10 seconds, then the leak may be determined to have failed and an indication to that effect may be rendered such as “LEAK TEST OF VALVE V1 HAS FAILED”.

FIG. 4H shows a portion of a block diagram of a system 400H that is similar to the system 400A of FIG. 4A operating in accordance with embodiments of the present system. This figure illustrates a configuration that may be utilized to discharge the leak test sensor (e.g., the airway pressure sensor and/or leak test sensor reservoir) when a leak test is complete. In this configuration, the purge pump is off and any pressure in the leak test sensor reservoir may be discharged through V3 to atmosphere. In accordance with embodiments, in a case wherein the airway pressure does not measure near zero after discharge, then V3 is not opening correctly indicating a failure. In accordance with embodiments, an indication to that effect may be rendered such as “VALVE V3 HAS FAILED”. In accordance with embodiments, in a case wherein the differential pressure sensor does not measure near zero after discharge, then V4 (shunt valve) is not opening correctly indicating a failure. In accordance with embodiments, an indication to that effect may be rendered such as “VALVE V4 HAS FAILED”.

FIG. 5 shows a functional flow diagram performed by a process 500 in accordance with embodiments of the present system. The process 500 may be performed using one or more computers communicating over a network and may obtain information from, and/or store information to one or more memories which may be local and/or remote from each other. The process 500 may include one of more of the following acts. In embodiments, the acts of the process 500 may be performed using one or more suitable gas monitoring systems such as a side-stream or main-stream monitoring system (SSM) or the like operating in accordance with embodiments of the present system. Further, one or more of these acts may be combined, reordered and/or separated into sub-acts, if desired. Further, one or more of these acts may be skipped depending upon settings. In operation, the process may start during act 501 and then proceed to act 503.

During act 503 the process may determine whether an accessory such as a patient-type interface or test-type interface (e.g., which may be respectively similar to the patient-type and test type interfaces 150 and 160, respectively) are coupled to a flow receptacle portion (e.g., 104, 304, 404) of a gas flow portion (e.g., 102). Accordingly, in a case wherein it is determined that the accessory is coupled to the flow receptacle of the gas flow portion, the process may continue to act 505. However, in a case wherein it is determined that an accessory is not coupled to the flow receptacle of the gas flow portion, the process may repeat act 503. In accordance with embodiments of the present system, the process may determine that an accessory is coupled to the flow receptacle portion using any suitable method such as by recognizing an ID of the accessory, by determining resistance of the accessory, querying (e.g., interrogating) an RFID chip of an accessory, mechanical keying methods, etc. For example, in accordance with embodiments, the process may control an RFID interrogator to query an RFID ID code from the accessory when it is coupled to, or in close proximity to, the gas flow portion. In yet other embodiments, mechanical sensors or optical code readers may be employed to read an ID of an accessory coupled to a gas flow portion. During act 505, the process may determine an operating mode based upon the ID of the accessory identified during act 503. For example, the ID may include identification of the type of accessory such as identifying whether the accessory is a patient-type interface or a test-type interface.

In accordance with embodiments of the present system, IDs may identify one or more settings (e.g., default, configuration, operating parameters, timing, operative acts, etc.) of an associated interface (e.g., as may be set by the system, process and/or user). Further, these settings may define operative acts to be performed by the process. For example, an operation time of a purge pump and/or operating pressures may be dependent upon settings defined in the ID of an accessory for example, that may account for reservoir capacity of the accessory, etc. In this way, in accordance with embodiments of the present system, operating characteristics (e.g., test parameters, operational parameters, etc.) may be defined by ID of an accessory.

Accordingly, an accessory type (e.g., patient-type interface or test-type interface) may have corresponding operative steps (e.g., breath-sensing, test-mode, respectively) assigned thereto. For example, in a case wherein the process identifies a patient interface as being attached to a gas module, (e.g., which may be recognized according to its ID), the process may then determine an operating mode based upon the recognized ID.

In accordance with embodiments of the present system, each operating mode may have corresponding mode information. For example, the process may obtain mode information associated with the recognized ID from a memory of the system and perform one or more acts accordingly. For example, in accordance with embodiments, the process may obtain information stored in a mode table including threshold pressure levels, etc., to determine acts to perform and/or parameters that are utilized during the acts. Mode tables operating in accordance with embodiments of the present system may be set and/or reset by the system, process and/or user and may be stored in a memory of the system for later use, as desired. In accordance with embodiments of the present system, upon determining an interface's type, the process may obtain mode information (e.g., from a mode table) for the corresponding interface type and perform one or more acts in accordance with the corresponding mode information. The process may further form a user interface (UI) which may render mode information (e.g., as may be stored in the mode tables) and/or with which a user may interact to edit the mode information (e.g., a menu-based UI). Then the edited (e.g., set/reset mode information) may be stored in a memory of the system for later use.

In accordance with embodiments of the present system, prior to operating in one or more of the configurations above (e.g., purge, zero, etc.), a process may verify whether the leak test sensor (e.g., leak-test accessory) is properly connected. For example, the process may determine whether a positive connection is established by, for example, reading an ID of an accessory (e.g., 106) coupled to a gas flow module.

Further, in a case wherein a patient connection is detected by the process, the process may set an error indication when breaths are detected (e.g., leak testing should not be performed). For example, the error indication may indicate that a patient is connected to the interface which is not desired during testing. The process may then render information related to the error indication to inform a user of results of the process and/or provide a recommendation such as: “PATIENT CONNECTED—TEST MODE CANNOT BE PERFORMED WHILE PATIENT CONNECTED,” or “BREATHS DETECTED, PLEASE CONFIGURE FOR TEST MODE TO RUN TEST,” or other information as may be set by the system and/or user. A patient connection may be detected when it is determined that airway flow is detected or otherwise measured (e.g., by pressure changes) by the system, either in the inspiratory (positive) and/or expiratory (negative) directions.

In accordance with embodiments, in a case wherein a patient connection is not detected, the process may continue to operate in accordance with the examples discussed herein. For example, an ID of an accessory may be analyzed to determine a type of accessory such as whether an accessory is a patient accessory or a test accessory, etc.

With reference to one or more acts of the sequence (e.g., one or more of the test sequences, such as illustratively shown in FIGS. 4F-4H) may form at least a portion of a leak-test sequence that may be able to test (e.g., check) elements of a corresponding gas flow module such as a Respironics FloTrak Elite™ module available from Philips Respironics. However, it is envisioned that one or more acts and/or sequences of the test sequences may be formed similarly for other makes and/or models of gas flow modules. In accordance with embodiments of the present system, one or more test sequences may be formed to determine one or more operating conditions such as one or more of:
a) Leak rate and pressure performance of an on-board purge pump of a gas flow portion (e.g., a gas flow module) may be determined in accordance with embodiments of the present system. For example, in a case wherein the purge pump leaks or is not operating properly following suitable operation of valves, purge pump, etc., the purge pump may not be able to attain a required test pressure such as a leak-test threshold pressure within one or more portions of the pneumatic system. Accordingly, the system may analyze sensor information to determine pressure within pneumatic system to detect this condition in accordance with embodiments of the present system. For example, in a case wherein a threshold pressure within the pneumatic system is not attained and/or maintained as compared to an attained pressure within/for a predetermined time period, the system may determine that the purge pump is malfunctioning or the pneumatic system is leaking and thereby, provide an indication to that effect.
b) Patient side and vent side performance embodiments of the present system may determine whether the patient side and vent side pneumatic systems are leaking through an analysis of pressure sensor information following suitable operation of valves, purge pump, etc. There should be no leaks to atmosphere from the flow receptacle following suitable operation of valves, purge pump, etc. For example, in a case wherein a threshold pressure within the patient side and vent side pneumatic systems is not attained and/or maintained as compared to an attained pressure within/for a predetermined time period, the system may determine that the patient side and vent side pneumatic systems are leaking. Pressure may be detected over time by sensors such as pressure transducers of the gas flow portion which may provide pressure information and thereby, provide an indication to whether the patient side and vent side pneumatic systems are leaking.
c) Patient and purge valves may be suitably operated to determine whether there are any leaks from the patient side valve and/or the purge valve V2 following suitable operation of valves, purge pump, etc. Accordingly, the system may analyze sensor information to determine pressure within pneumatic system to detect this condition in accordance with embodiments of the present system. For example, in a case wherein a threshold pressure within the pneumatic system is not attained and/or maintained as compared to an attained pressure within/for a predetermined time period, the system may determine that the patient side valve and/or the purge valve is leaking and thereby, provide an indication to that effect.
d) V3 (atmosphere valve)—the system may determine whether there is a leak thru this valve to atmosphere. Accordingly, the system may analyze sensor information to determine pressure within pneumatic system to detect this condition in accordance with embodiments of the present system following suitable operation of valves, purge pump, etc. For example, in a case wherein the atmosphere valve V3 is open so as to not provide a fluid path to atmospheric pressure, it is determined whether a threshold pressure with the atmosphere valve V3 is attained and/or maintained as compared to an attained pressure within/for a predetermined time period. In this way, the system may determine whether the atmosphere valve V3 is malfunctioning or the pneumatic system is leaking and thereby, provide an indication to that effect.

As may be readily appreciated, through operation of the valves, pumps, etc., different portions may be operated and detected to determine whether the different portions are operating within desired operating parameters (e.g., such as attaining and/or maintaining a pressure within/for a predetermined time period. In accordance with embodiments of the present system, a sequence of tests may be utilized to determine whether a giving portion is operating as desired. For example, in a case wherein a purge test has been successfully completed, the process may subsequently test other portions of the system and in case of failure, produce an indication of a failed portion excluding portions that have already passed prior tests.

In a case wherein any leaks are detected, the system may set a fail flag which may indicate that the gas flow portion (e.g., gas flow module) has failed a current test, may also provide an indication of particular portions that have failed and may store results of the test for later use and/or may provide an indication immediately following a failure or some other subsequent time. For example, the process may provide an indication such as when the gas flow portion is subsequently utilized such as setup prior to intended patient support through the “failed” gas flow portion. In this way, the process may detect these flags at a later time such as when starting a patient mode (e.g., a breathing analysis mode) so that proper action may be taken such as termination of the corresponding mode so as to avoid analysis errors and/or improper patient support such as attempted use of the “failed” gas flow portion.

Although only two different types of interfaces are shown, it is envisioned that in accordance with embodiments of the present system, more than two-different types of interface types may be employed. Accordingly, one or more of these different interface types may have corresponding mode information that may be defined by the user and/or system and which may be stored in a memory of the system for later use. In accordance with embodiments, the mode information may be stored and obtained from the corresponding accessory in which case this information may be used to identify an accessory as previously discussed.

In accordance with embodiments of the present system, an operating mode, operation, etc., may be selected by a user. Accordingly, the process may form a user interface with which a user may interact to select an operating mode, operation, etc. The process may then determine to use this operating mode, operation, etc.

After completing act 505 (e.g., determining the operating mode), the process may continue to act 507 during which, the process may be operative to perform the acts in accordance with the corresponding mode, etc. that was determined during act 505. Accordingly, the process may control one or more portions of a gas flow portion (e.g., circuits, valves, pumps, sensors, etc. of, for example, PFPs 100, 400) in accordance with the determined operating mode. Accordingly, the process may perform the actions and the corresponding determinations (if applicable) as may be defined in the corresponding mode information for the identified mode. After completing act 507, the process may continue to act 509.

During act 509, the process may render for example on a rendering device of the system (e.g., a display, a speaker, etc.) information related to the determinations (e.g., results) of the process. For example, the process may render information indicating that a “pneumatic system test has passed” or the “pneumatic system test has failed” or portions thereof have passed/failed which may be dependent upon whether a test mode performed in accordance with embodiments of the present system has passed or failed, respectively. The messages may be set by the system, process and/or user and stored in memory of the system. As each process may vary based upon the operating mode (e.g., where the operating mode may be a test mode, breathing analysis mode, etc.), the information that may be rendered may vary depending upon the operating mode. For example, in a case wherein the operating mode is a breathing analysis mode, the process may render information related to, for example, characteristics of a sampled ventilation gas flow such as one or more of percent concentration, volume, flow, and pressure. However, in a case wherein the operating mode is a test mode, then the process may render information related to the test mode. For example, assuming that the process is test mode process, the process may render information such as a verification of whether a test accessory was successfully connected, whether breaths were detected, and/or a general pass/fail indication (e.g., test status information) of the pneumatic portion (or specific portions thereof) (e.g., valve (V4) leakage detected, etc.), etc. for the convenience of the user. For example, in accordance with embodiments, in a case wherein it is determined that the pneumatic system test was successful, the process may render information informing a user of what to do next such as “Pneumatic System Test Successful. Please Remove The Test Accessory and Insert Patient Accessory.” However, in a case wherein it is determined that the pneumatic system test was not successful, the process may render information that “the Pneumatic System Test has Failed, Please Remove Test Accessory and Reinsert,” provide some indication of which portions, such as valves, that have failed and/or the like. After completing act 509, the process may continue to act 511.

During act 511, the process may update a system history in accordance information related to the determinations of the process. The system history may be stored in a memory of the system for later use such as in a case wherein the gas flow portion is subsequently connected to the system, such as for subsequent patient support. In this way, a gas flow portion that has failed one or more of the tests, such as those described herein, may be subsequently flagged (e.g., provide an indication that the gas flow portion may be faulty) should an attempt be made to use the faulty gas flow portion, for example, for patient support. After completing act 511, the process may continue to act 513 where the process may end.

In accordance with embodiments of the present system, at the start of a breathing analysis mode, the process may access the history information to determine whether a breathing analysis was run within a threshold time period such as within the past 72 hours. Accordingly, in a case wherein it is determined that a breathing analysis mode was run within this threshold time period (e.g., 72 hours as may be set by the system, process and/or user), the process may continue the breathing analysis mode. However, in a case wherein it is determined that a breathing analysis mode was not run within the threshold time period, the process may inform a user to run the test analysis mode as soon as possible, such as prior to use. The process may further determine whether a most recently run test analysis mode was successfully run. Accordingly, in a case wherein it is determined that the most recently run test analysis mode was successful the process may continue to run the breathing analysis mode. However, in a case wherein it is determined that the most recently run test analysis mode was not successfully run (e.g., result=fail), the process may not continue to perform the current breathing analysis mode, for example without an override from a user. In accordance with embodiments of the present system, this may prevent use of the system absent an override in a case wherein the system is determined to have failed a most recently run test analysis mode absent an override (e.g., as may be provided by a menu-item (e.g., within a user interface (GUI) rendered by the system).

Further, in accordance with embodiments of the present system a method of use may include the following acts such as connecting a leak test accessory to a gas flow module; entering a request to activate a test mode, the request may be generated by the system (e.g., in response to determining that the leak test accessory is couple to a gas flow module as described herein) or by a user (e.g., in response to a user's request entered at a user interface of the system); then performing the test mode to determine whether the gas flow portion has passed or failed the test mode. The results of the test(s) (e.g., a pass/fail status) may then be stored in a memory of the system and/or rendered for the convenience of a user on a user interface (e.g., a display) of the system. The system may then render information requesting a user disconnect the leak test accessory. In accordance with embodiments of the present system, the system may not enter a gas analysis mode or may terminate a gas analysis mode in a case wherein it is determined that the system has failed a current leak test. In accordance with yet other embodiments, the system may not enter a breath analysis mode until the leak test accessory is disconnected from the gas flow portion. In accordance with yet other embodiments, only a single accessory may be coupled to the gas flow portion at a time.

FIG. 6A shows a block diagram of a portion of a system 600A operating in a ventilation detection mode in accordance with embodiments of the present system. More particularly, a gas flow module 602 may be coupled to a patient interface 603 via an accessory 606. The accessory 606 may be similar to the accessory 106 and may include proximal and distal flow receptacles 608 and 654, respectively, which may be respectively similar to the proximal and distal flow receptacles 108 and 154, respectively, of the accessory 106. The distal flow receptacle 654 may be coupled to the patient interface 603 and a ventilator 605 which may provide a ventilation gas to the patient interface 603. The patient interface 603 may be coupled to a patient 601 whom may be ventilated by the ventilation gas. The gas flow module 602 may obtain a sample gas flow from the distal flow receptacle 654 and analyze this gas to determine, for example, one or more of percent concentration, volume, flow, pressure of the ventilation gas and/or failure as described herein. This information may then be rendered on a display of the system for the convenience of the user and/or may be stored in a memory of the system for later use. Although a ventilator 605 is shown providing the ventilation gas, in yet other embodiments of the present system it is envisioned that the user may obtain the ventilation gas from any suitable source such as atmosphere.

For example, FIG. 6B shows a block diagram of a portion of a system 600B operating in a flow detection mode in accordance with embodiments of the present system. The system 600B may be similar to the system 600A. Accordingly identical numerals are provided for the sake of clarity. However, rather than obtaining the ventilation gas from a ventilator such as the ventilator 605 shown in FIG. 6A, the gas (VG) may be obtained from atmosphere.

It is envisioned that embodiments of the present system may monitor patients who are not ventilated (e.g., patients not connected to a ventilator but whose breathing is otherwise monitored). For example, embodiments of the present system may be used to provide respiration flow monitoring of non-ventilated patients such as respiration monitoring of athletes, respiration monitoring of cardiac patients undergoing stress tests, and the like. Accordingly, embodiments of the present system may be used to monitor respiration of a patient without support from a ventilator. In accordance with embodiments of the present system, other gas flow systems may be suitably utilized, such as a gas flow system utilized during patient testing.

Moreover, although embodiments of the gas flow module are shown situated at or near the patient, in accordance with embodiments of the present system it is further, envisioned that the gas flow module may be placed at other locations or otherwise situated. For example, the gas flow module in accordance with embodiments of the present system, the gas flow module may be remote from the patient, such as coupled through a gas conduit.

For example, FIG. 6C shows a block diagram of a portion of a system 600C operating in a ventilation detection mode in accordance with embodiments of the present system. The system 600C may be similar to the systems 600A and/or 600B. Accordingly, identical numerals are provided for the sake of simplicity. However, in accordance with embodiments of the present system the accessory 606 is flow coupled to the patient interface 603 via an extension tube 670 of a given length. Accordingly, the proximal flow module may be situated apart from the patient as desired. However, the gas flow module may take into account flow characteristics due to extended flow paths of the ventilation gas mixture which may be due to extended tubing, etc. when operating in accordance with embodiments of the present system. These characteristics may include, for example, gas compressibility, tubing distortion, etc.

FIG. 7 shows a portion of a system 700 in accordance with embodiments of the present system. For example, a portion of the present system may include a processor 710 (e.g., a controller) operationally coupled to a memory 720, a rendering device such as a display 730, sensors 740, actuators 760, a network 780, and a user input device 770. The memory 720 may be any type of device for storing application data as well as other data related to the described operation. The application data and other data are received by the processor 710 for configuring (e.g., programming) the processor 710 to perform operation acts in accordance with the present system. The processor 710 so configured becomes a special purpose machine particularly suited for performing in accordance with embodiments of the present system. The actuators 760 may include actuators such as solenoids and/or motors which may control one or more valves and/or pumps of the system in accordance with embodiments of the present system.

The user input 770 may include a keyboard, a mouse, a trackball, or other device, such as a touch-sensitive display, which may be stand alone or be a part of a system, such as part of a personal computer, a personal digital assistant (PDA), a mobile phone (e.g., a smart phone), a monitor, a wearable display (e.g., smart glasses, etc.), a smart- or dumb-terminal or other device for communicating with the processor 710 via any operable link. The user input device 770 may be operable for interacting with the processor 710 including enabling interaction within a user interface (UI), GUI, etc., as described herein. Clearly the processor 710, the memory 720, display 730, and/or user input device 770 may all or partly be a portion of a computer system or other device such as a client and/or server type device.

The actuators 760 may include one or more motors, transducers, etc. which may provide a force or power to operate one or more valves, pumps, mixers, or the like of the SSM 160 under the control of the processor 710. These valves may, for example, include pneumatic control valves which may control the flow of one or more gasses for ventilation, etc.

The methods of the present system are particularly suited to be carried out by a computer software program, such program containing modules corresponding to one or more of the individual steps or acts described and/or envisioned by the present system. Such program may of course be embodied in a computer-readable medium, such as an integrated chip, a peripheral device or memory, such as the memory 720 or other memory coupled to the processor 710.

The program and/or program portions contained in the memory 720 may configure the processor 710 to implement the methods, operational acts, and functions disclosed herein. The memories may be distributed, for example between the clients and/or servers, or local, and the processor 710, where additional processors may be provided, may also be distributed or may be singular. The memories may be implemented as electrical, magnetic or optical memory, or any combination of these or other types of storage devices. Moreover, the term “memory” should be construed broadly enough to encompass any information able to be read from or written to an address in an addressable space accessible by the processor 710. The memory 720 may include a non-transitory memory. With this definition, information accessible through a network such as the network 780 is still within the memory, for instance, because the processor 710 may retrieve the information from the network 780 for operation in accordance with the present system.

The processor 710 is operable for providing control signals and/or performing operations in response to input signals from the user input device 770 as well as in response to other devices of a network and executing instructions stored in the memory 720. The processor 710 may include one or more of a microprocessor, an application-specific or general-use integrated circuit(s), a logic device, etc. Further, the processor 710 may be a dedicated processor for performing in accordance with the present system or may be a general-purpose processor wherein only one of many functions operates for performing in accordance with the present system. The processor 710 may operate utilizing a program portion, multiple program segments, or may be a hardware device utilizing a dedicated or multi-purpose integrated circuit.

The methods of the present system are particularly suited to be carried out by processor programmed by a computer software program, such program containing modules corresponding to one or more of the individual steps or acts described and/or envisioned by the present system.

Accordingly, embodiments, of the present system may provide an automated fixture that can detect multiple failure modes as compared to a single failure mode in conventional manual fixtures. For example, an advantage of embodiments of the present system is the ability to detect when an internal purge pump is malfunctioning which is generally a failure mode that is undetectable by the existing fixtures that are utilized for providing ventilation to a patient and/or for monitoring a patient. Further, embodiments of the present system may simplify field servicing pneumatic systems with automated repeatable tests to check for system leaks as well as the performance of a purge pump. Further, embodiments of the present system may provide for field servicing which may test features of a patient based pneumatic system without the use of an external pressure source, pressure meter, leak tester, and/or flow meter. In accordance with embodiments of the present system multiple (e.g., such as 6) failure modes may be detected and corresponding information rendered for the convenience of a user.

It is further envisioned that embodiments of the present system may be used in with critical-care ventilators, home ventilators, and the like. Further, it is envisioned that embodiments of the present system may be used in various medical environments such as intensive-care units (ICU), operating rooms (OR), emergency departments, ambulatory care, doctors' offices, stress testing offices, and/or other facilities where respiratory gases may be provided to a patient and/or a patient's respiratory state may be accessed.

Finally, the above-discussion is intended to be merely illustrative of the present system and should not be construed as limiting the appended claims to any particular embodiment or group of embodiments. Thus, while the present system has been described with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function;
e) any of the disclosed elements may be comprised of hardware portions (e.g., including discrete and integrated electronic circuitry), software portions (e.g., computer programming), and any combination thereof;
f) hardware portions may be comprised of one or both of analog and digital portions;
g) any of the disclosed devices or portions thereof may be combined together or separated into further portions unless specifically stated otherwise;
h) no specific sequence of acts or steps is intended to be required unless specifically indicated;
i) the term “plurality of” an element includes two or more of the claimed element, and does not imply any particular range of number of elements; that is, a plurality of elements may be as few as two elements, and may include an immeasurable number of elements; and
j) the term and/or and formatives thereof should be understood to mean that only one or more of the listed elements may need to be suitably present in the system in accordance with the claims recitation and in accordance with one or more embodiments of the present system.

1. A gas flow monitoring system for monitoring a patient gas flow and having at least one port, pressure sensors, a pneumatic system, valves, and a pump, the system comprising:
a controller configured to:
configure the pump and the valves to pressurize at least a portion of the pneumatic system when in a test mode;
obtain sensor information indicating pressure within at least a portion of the pneumatic system, when in the test mode;
determine whether a leak test fails based upon at least the sensor information; and
determine whether an accessory is coupled to the at least one port, the accessory including at least one of a patient-type interface or a test-type interface.

2. The gas flow monitoring system of claim 1, wherein the controller is configured to render results of the determination indicating whether the leak test has failed.

3. The gas flow monitoring system of claim 1, wherein the at least one port comprises a gas flow portion that is proximal to the patient.

4. (canceled)

5. The gas flow monitoring system of claim 1, wherein the controller is configured to determine a type of accessory when it is determined that an accessory is coupled to the at least one port.

6. The gas flow monitoring system of claim 1, wherein the accessory comprises identification information which identifies a type of the accessory.

7. The gas flow monitoring system of claim 6, wherein the controller is configured to read the identification information from the accessory portion.

8. The gas flow monitoring system of claim 7, wherein the controller is configured to identify a type of accessory based upon the identification information.

9. The gas flow monitoring system of claim 8, wherein the controller is configured to determine that the test mode is selected when the type of accessory is determined to be a test type.

10. The gas flow monitoring system of claim 8, wherein the controller is configured to determine whether a breath analysis mode is selected based on the identified type of accessory.

11. The gas flow monitoring system of claim 10, wherein the controller is further configured to control the pump and the valves to obtain a sample gas flow from a patient interface when it is determined that a breath analysis mode is selected.

12. The gas flow monitoring system of claim 1, wherein the controller is configured to render results of the determination of whether the leak test fails.

13. The gas flow monitoring system of claim 10, wherein when the breath analysis mode is selected, the controller is configured to determine whether a recent test mode has previously failed.

14. The gas flow monitoring system of claim 13, wherein the controller is configured to terminate the breath analysis mode when it is determined that the recent test mode has previously failed.

15. A method for monitoring a patient gas flow in a system having at least one port, pressure sensors, a pneumatic system, valves, and a pump, the method comprising acts of:
configuring the pump and the valves to pressurize at least a portion of the pneumatic system when in a test mode;
obtaining sensor information indicating pressure within at least a portion of the pneumatic system, when in the test mode;
determining whether a leak test fails based upon at least the sensor information;
determining whether an accessory is coupled to the at least one port; and
determining a type of accessory when it is determined that an accessory is coupled to the at least one port, the accessory including at least one of a patient-type interface or a test-type interface.

16. (canceled)

17. The method of claim 15, comprising an act of determining that the test mode is selected when the type of accessory is determined to be a test type.

18. A computer readable non-transitory medium having computer readable program code for operating on a computer for performing a method of monitoring a patient gas flow in a system having at least one port, pressure sensors, a pneumatic system, valves, and a pump, the method comprising acts of:
configuring the pump and the valves to pressurize at least a portion of the pneumatic system when in a test mode;
obtaining sensor information indicating pressure within at least a portion of the pneumatic system, when in the test mode;
determining whether a leak test fails based upon at least the sensor information;
determining whether an accessory is coupled to the at least one port; and
determining a type of accessory when it is determined that an accessory is coupled to the at least one port, the accessory including at least one of a patient-type interface or a test-type interface.

19. (canceled)

20. The medium of claim 18, the method comprising an act of determining that the test mode is selected when the type of accessory is determined to be a test type.
